(19) United States
(12) Patent Application Publication
(10) Pub. No.: US 2007/0141568 A1
(43) Pub. Date: Jun. 21, 2007

(54) Method for determining the presence of one or more ligands in a sample

(75) Inventors: Bart Van Der Burg, 't Goy (NL); Edwin Sonneveld, Hoek van Holland (NL); Abraham Brouwer, Wassenaar (NL)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: BioDetection Systems B.V.

(21) Appl. No.: 10/539,455

(22) PCT Filed: Dec. 22, 2003
(86) PCT No.: PCT/EP03/14813

§ 371 Date: Dec. 13, 2006

(30) Foreign Application Priority Data
Dec. 20, 2002 (EP) PCT/EP02/14877

(51) Int. Cl. C12Q 1/68; G01N 33/567; C12N 5/08

(52) U.S. Cl. 435/6; 435/366; 435/7.2

(57) ABSTRACT

The present invention relates to a method for determining the presence of one or more specific ligands in a sample, said method comprising: a) contacting the sample with an array of cell lines, each cell line comprising a reporter gene construct responding to a cellular pathway which is induced by a different specific ligand; b) measuring the activity of the reporter gene in the individual cell lines; c) comparing the measured activity in the individual cell lines; and d) determining the presence of the ligands in the sample based upon said comparison.

The present invention relates to a method for determining the presence of one or more ligands in a sample.

Identification of biologically active compounds, i.e. ligands, is important in a variety of fields, such as for example pharmacological and clinical screening, food manufacturing and toxicological monitoring of compounds. Traditionally, the monitoring strategies focus on two extreme methods: i.e., sophisticated detailed chemical analysis, and determination of biological effects using whole animal assays, and epidemiology. With those methods a correlation can be made between tissue or environmental levels of a compound and the effect seen in an organism (exposure and effect determinations).

Major problems occur when chemical analysis is inadequate to predict biological effects because of metabolism of the parent compound to metabolites with unknown biological activity, or when complex mixtures of biologically active compounds are present, which is generally the case in environmental and food samples. In addition, the determination of biological effects in experimental animals has various limitations. First, the tests are costly and time consuming. Additionally, a limited number of endpoints can be monitored in a single animal, and because of tissue and species-specific responses to compounds the predictions made often are inaccurate. Moreover, there is an increasingly strong pressure to reduce the amount of animal testing.

With many new chemicals, food additives and pharmaceuticals entering the market each year, there is a need for high throughput testing methods. The revolution in molecular biology and biotechnology has indeed allowed development of a broad spectrum of novel high speed testing strategies using cultured cells, isolated biological endpoints, such as receptors and other biological molecules, and genetically modified organisms.

Biological endpoints that have a priority in risk assessment of chemicals are carcinogenicity, mutagenicity and reproductive toxicity (‘CMR” substances; European Commission (2001), White Paper, Strategy for a future chemicals policy, Brussels) or POP (persistent organic pollutants) characteristics. In reproductive toxicity emphasis has currently been laid on chemical interfering with androgen, estrogen and thyroid hormone action.

Steroid hormones are essential in most reproductive processes and can influence many other physiological processes as well. Due to the relatively simple chemical structure and lipophylic nature of steroids, their cellular regulatory pathways can be easily modified by pharmacological, environmental- and dietary ligands. As a consequence, steroids and steroid-mimicking compounds find applications in many fields, and their detection is important in a range of fields, for example in doping control, meat quality control, medical practice, environmental and food monitoring, etc.

Steroid hormones enter cells by diffusion whereafter they bind to intracellular receptors. Five major types of receptors are known: receptors for estrogens, androgens, progestins, glucocorticoids and mineralocorticoids. Upon ligand binding the receptors become activated, whereafter they enter the nucleus and bind to recognition sequences in promoter regions of target genes, i.e. the hormone responsive element. The DNA bound receptor will activate transcription of the target gene. This will lead to new protein synthesis and an altered cellular functioning.

Recently a screening method for estrogenic compounds has been developed making use of the fact that the receptor for estrogens is a transcription factor that induces transcription of target genes after binding to specific DNA sequences in their promoter. When these DNA sequences are linked to the gene of an easily measurable protein (the so-called “reporter gene”) and introduced in a suitable cell line, an estrogen responsive reporter cell line can be generated allowing large scale screening of for example chemicals. A highly selective and responsive reporter gene construct with exceptionally low background activity was generated in which three estrogen responsive elements were coupled to a very minimal promoter and luciferase. Upon stable introduction in human T47D breast carcinoma cells a highly sensitive biological detection method was generated (Legler et al., Toxicol. Sciences 48: 55-66, 1999). Although this method is suitable for determining the estrogen activity of specific compounds, this method does not provide information on the presence of other unknown ligands in samples comprising a complex mixture of different biologically active compounds.

The object of the present invention is to provide a simple and reliable method for determining the presence of one or more ligands in a sample.

This object is achieved by the invention by a method comprising:

a) contacting the sample with an array of cell lines, each cell line comprising a reporter gene construct responding to a cellular pathway which is induced by a different specific ligand;

b) measuring the activity of the reporter gene in the individual cell lines;

c) comparing the measured activity in the individual cell lines; and

d) determining the presence of the ligands in the sample based on said comparison.

The method according to the present invention thus allows rapid in vitro screening of samples for the presence of specific unknown ligands which may have potentially beneficial or, more importantly, toxic effects. The sample may for example be a biological sample, such as blood, plasma, serum or other bodily fluids (e.g. to test for steroid abuse), environmental samples, or food samples, etc. The sample may also comprise single chemical compounds. The method is simple, reliable, rapid and animal friendly, while still covering an array of biological endpoints that may be targeted by the ligands, such as chemical compounds. The method is highly sensitive, and is suitable in quality control procedures since the method also detects the effects of (intentionally) masked compounds or complex mixtures thereof, which escape the conventional chemical detection. Moreover, the method allows measurement of the activity of compounds, regardless of their chemical nature. The method is in particular suitable for first line screening, directing further decisions on more extensive risk analysis using animals in the case of effect assessment of known compounds or compound identification, and/or using chemical analysis in the case of quality control of environmental, food or biological samples.

To set up an array of cell lines for use in the method according to the invention, it is advantageous to use a single cell line as the recipient, i.e. to have the cell lines originating from one parent cell line. This will allow a better comparison between the individual cell lines of the system and detection of non-specific effects.

Prior to the invention a cell line in which all steroid receptors are highly active was unknown. In the research that led to the present invention is has surprisingly been found that the human bone cell line U2-OS can suitably be used to make highly responsive cell lines for a whole range of receptors, including some of the ‘difficult’ ones like the androgen receptor. Given the known effects of thyroid hormone on osteoblastic bone cells, this cell line may also be used to make a thyroid hormone responsive cell line. According to a preferred embodiment of the present invention the cell lines therefore originate from the human osteoblastic cell line U2-OS.

The array of cell lines preferably comprises at least two cell lines, more preferably at least three cell lines. Even when using such relatively small array of cell lines, it is to be expected that for example all steroid ligands test positive in one or more of the cell lines, thereby minimizing the occurrence of false-negative results. The pattern of activation gives an indication of what type of steroid might be present in the sample.

According to a preferred embodiment of the method, one or more of the cell lines comprise one or more expression plasmids each coding for a specific component of the cellular pathway, for example in case the cell line does not endogenously express said component. The term “component” herein refers to an element (i.e. receptor, enzyme, second messenger) which is part of a specific intracellular signalling pathway that is induced by a specific ligand. Preferably, the specific component is a hormone receptor, more preferably a steroid hormone receptor (i.e. androgen receptor, estrogen receptor (alpha and beta), progesterone receptor, glucocorticoid receptor, mineralocorticoid receptor) or thyroid hormone receptor.

It is known that metabolism of steroids can substantially alter their biological properties, and that enzymatic conversions can lead to steroids with a totally different activity profile. Therefore, with a highly specific test method metabolism may complicate interpretation of the results. However, since metabolism also occurs in vivo metabolic steps may be beneficial to include in the test method since these can give a level of integration which will enhance the predictability of the in vivo response. For example, high concentrations of non-prohibited precursor steroids or precursor steroids that are difficult to trace are used in sport doping, rather than the active hormone itself. When the precursor is converted into the active hormone at the level of the target cell, as often is the case, a mechanism of detection (either chemical of biological) directed towards the active circulating hormone will be ineffective. For instance, an androgen assay that does not detect these precursors may give a high level of false-negative results. In contrast, with the method of the present invention, by providing an array of cell lines which are responsive to different hormones and precursors, the number of potential ligands that are measured by the method is increased.

According to another preferred embodiment the specific component is a ligand modifying factor. Preferably, the ligand modifying factor is an enzyme. Thus, the discriminative power of the method of the invention is further enhanced by including cell lines with a modulated pattern of enzyme activity. It has for example been found that the 3-β HSD (hydroxysteroid dehydrogenase) enzyme of which the activity includes conversion of DHEA in androstenedione may be inducible in the androgen-responsive cell line by interleukins, thereby enhancing the range of active compounds and discriminative power of the method. It is also possible to modulate the metabolic activity of one or more of the cell lines by pharmacological manipulation, leading to expression of genes that are endogenously contained in the genome of the cell lines. An alternative way of introduction of metabolic capacity is using co-cultures with cells that express thus enzyme, or adding (partially) purified enzymes. The cell lines used may further comprise one or more other components for additional cellular signalling pathways involved in biological responses of toxicological and/or pharmacological concern, such as apoptosis and/or cell death, cytokine, stress, DNA-damage, growth factor inducible transcriptional responses (NF-kappaB, AP-1, STAT, p53), retinoid and dioxin receptor inducible pathways.

According to a preferred embodiment, the reporter gene construct comprises DNA coding for an operative hormone responsive element linked to a promoter and a reporter gene. The reporter gene constructs according to the invention may for example comprise specific multimerized responsive elements which are cloned upstream of the synthetic TATA box in the pGL3-tata-Luc vector. Since the androgen receptor (AR), glucocorticoid receptor (GR), progesterone receptor (PR) and mineralocorticoid receptor (MR) recognize the same DNA sequence, the same reporter gene construct may be used for these receptors.

The reporter construct preferably comprises 3 tandem repeats of the hormone responsive element (HRE) oligonucleotide:

AAGCTTAGAACAGTTTGTAACGAGCTCGTTACAAACTGTTCTAGCTCGTTACAAACTGTTCTAAGCTCAAGCTT

upstream of the minimal adenovirus E1B TATA promotor sequence (GGGTATATAAT) inserted in the multiple cloning site of the luciferase reporter construct pGL3 (as described in Legler et al., supra). The DNA coding for the different receptors preferably is introduced in the pSG5 expression plasmid.

The presence of one or more ligands in the sample is determined by measuring the transcription of the reporter gene, for example the luciferase gene, in the individual cell lines. The transcribed luciferase protein will emit light when a suitable substrate is added. The amount of emitted light is directly related to the amount of the ligand. By comparing the measured activity in said individual cell lines, a profile of biological activities is found which is used for determining the presence of one or more ligands in the sample based on said comparison. Based on the profile it is possible to make a prediction on the expected biological activity of the sample, i.e. its expected toxicological, pharmacological and/or nutritional properties.

According to the present invention the term “ligand” refers to any chemical or biological compound that may be present in a sample. Ligands may for example include hormones, precursors or derivatives thereof, i.e. compounds which are directly derived from said hormones, such as metabolites which are produced for example by endogeneous enzymes, or analogues of said hormones, i.e. (chemical) compounds which does not belong to the class of hormones or derivatives thereof but which do bind to the hormone receptor and exert hormonal activity (hormone-mimicking ligands).

The present invention further relates to a human osteoblastic U2-OS cell line, comprising a reporter gene construct comprising DNA coding for an operative hormone responsive element linked to a promoter and a reporter gene, and one or more expression plasmids comprising DNA coding for a hormone receptor, wherein the hormone receptor is selected from the group consisting of androgen receptor, progesterone receptor, glucocorticoid receptor mineralocorticoid receptor, and thyroid receptor.

Furthermore, the invention relates to the use of a human osteoblastic cell line in an assay for determining the presence of one or more ligands in a sample. Preferably, the cell line is the U2-OS cell line. The extreme responsiveness of the receptors in these cells, in particular of steroid receptors, makes these cells not only suitable for use in the method of the present invention, but also as an individual cell line for determining specific single endpoints.

The invention is further illustrated by the following Example and Figures.

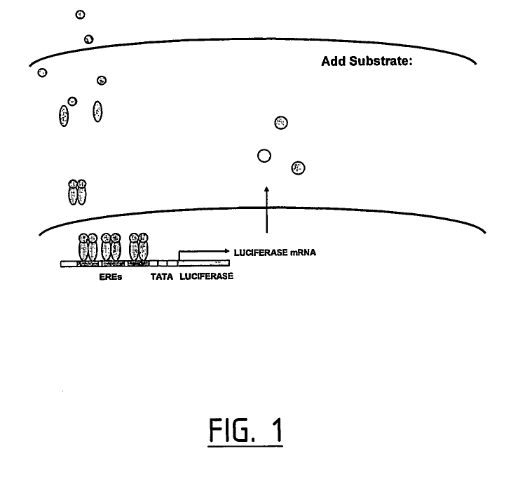

FIG. 1 schematically shows the principle of the detection of binding of an estrogen hormone or estrogen-mimicking ligand to the reporter gene construct according.

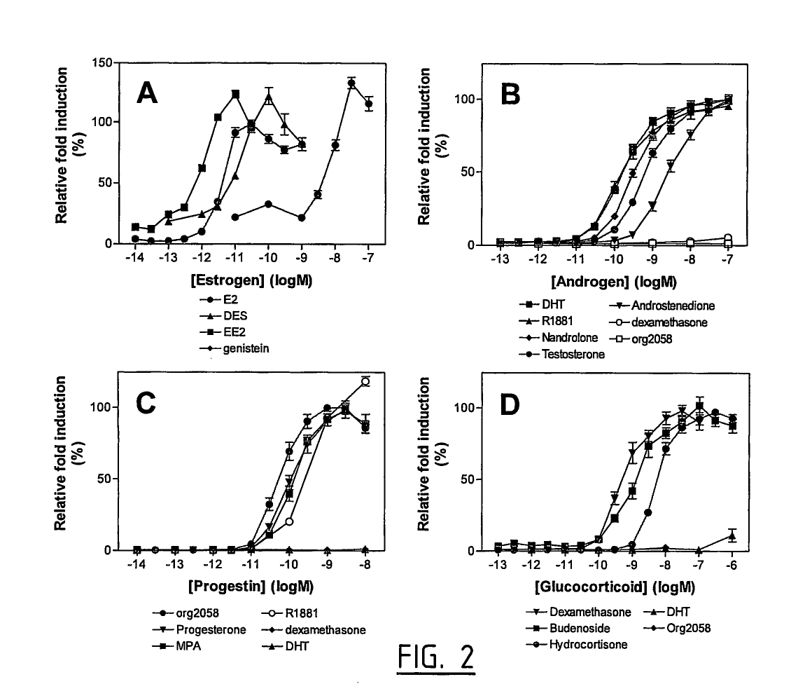

FIG. 2 shows four graphs wherein the responsiveness of four different U2-OS cell lines is demonstrated. A: U2-OS cell line comprising estrogen-alpha receptor; B: U2-OS cell line comprising androgen receptor; C: U2-OS cell line comprising progesterone receptor; D: U2-OS cell line comprising glucocorticoid receptor. Abbreviations used: E2: 17β-estradiol; DES: diethyl-stilbestrol; DHT: 5α-dihydro-testosterone; MPA: medroxyprogesterone acetate.

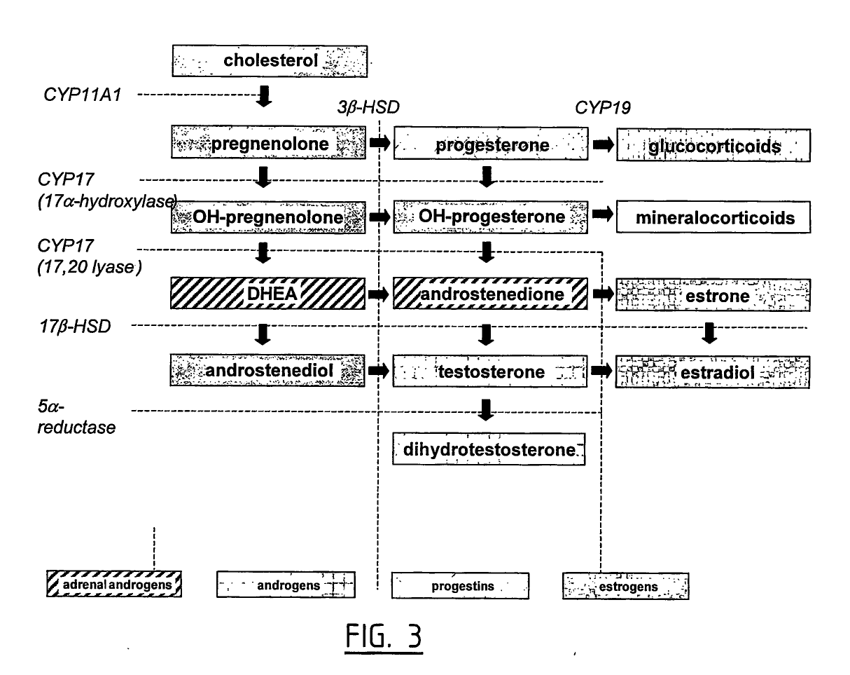

FIG. 3 is a scheme showing the different metabolic steps in the biosynthesis of steroid hormones. Steroid hormones are generated through a metabolic pathway in which small molecular changes are effected in each enzymatic conversion. Precursor molecules for one receptor type are specific hormones for other receptors.

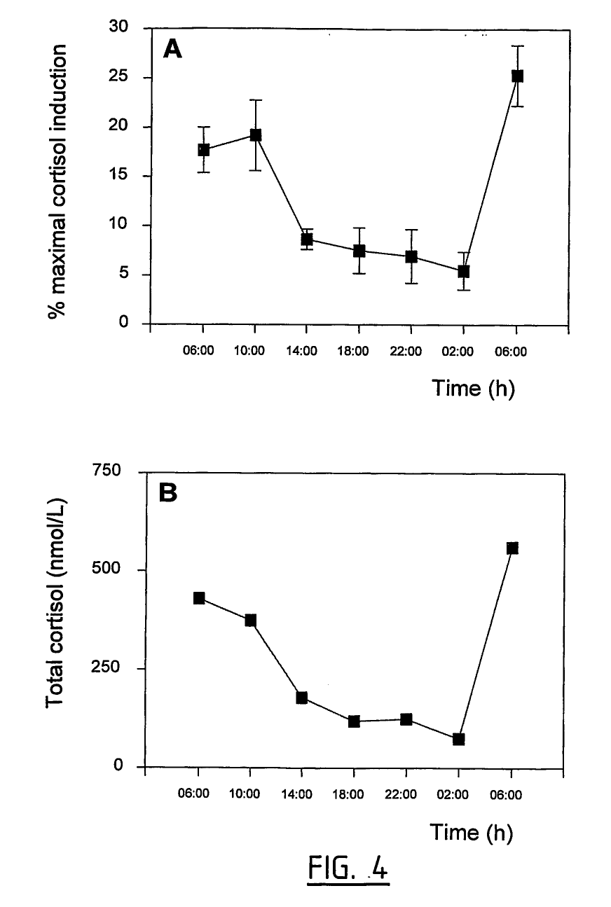

FIG. 4 shows the results of the U2-OS cell line comprising glucocorticoid receptor in measuring diurnal endogenous cortisol levels in human serum, compared to measurements by cortisol specific RIA.

### EXAMPLES

### Example 1

Suitability of U2-OS Cells to Generate Reporter Cell Lines for Steroid Receptors Using TATA-Luc Reporters

Reporter constructs were made using specific synthetic multimerized hormone responsive elements which were cloned upstream of the synthetic TATA box in the pGL3-TATA-Luc vector. Stable transfected cell lines were made, when necessary co-transfected with a specific receptor-containing expression plasmid pSG5.

FIG. 1 schematically shows the principle of the detection of binding of an estrogen hormone or estrogen-mimicking ligand to the reporter gene construct. Upon estrogen binding the estrogen receptor (ER) becomes activated and binds to the recognition sequences in promotor regions of target genes, the so-called estrogen responsive elements (EREs). Three of these EREs have been linked to a minimal promotor element (the TATA box) and the gene of an easily measurable protein (in the present case luciferase). The ligand activated receptor will activate luciferase transcription and the transcribed luciferase protein will emit light when a suitable substrate is added. The signal will dose-dependently increase with increasing concentrations of ligand.

Table 1 shows the suitability of U2-OS cells compared to other cell lines.

TABLE 1

| Cell line | ER | AR | PR | GR |
|---|---|---|---|---|
| T47D | • | −− | • | −− |
| HEK293 | • | −− | + | • |
| U2-OS | • | •• | • | •• |

Unsuitable (−−) because of failure to get receptors expressed; Suitable (+) as determined in transients transfections; (•) Suitable as demonstrated in stable transfections; (••) Exceptionally responsive cell lines (low EC50).

In the research that let to the present invention, it was found that the U2-OS cell line is particularly suitable to be used in the method according to the invention (Table 1). U2-OS cell lines supported the signalling pathways of many important ligands, such as steroid hormones. Efficient stable transfection is possible allowing easy expansion of the range of reporter cell lines. Moreover, the cells have been shown to be robust and to withstand routine handling in non-specialized laboratories. The generation time is low and rapid propagation is possible in conventional serum-containing media. The cells attach readily to standard culture materials, and remained stably transfected during more than 20 passages.

DNA Constructs

Since the androgen receptor (AR), glucocorticoid receptor (GR), progesterone receptor (PR) and mineralocorticoid receptor (MR) recognize the same DNA sequence, the same reporter gene construct was used for these receptors.

The reporter construct comprises 3 tandem repeats of the hormone responsive element (HRE) oligonucleotide:

AAGCTTAGAACAGTTTGTAACGAGCTCGTTACAAACTGTTCTAGCTCGTTACAAACTGTTCTAAGCTCAAGCTT

upstream of the minimal adenovirus E1B TATA promotor sequence (GGGTATATAAT) inserted in the multiple cloning site of the luciferase reporter construct pGL3 (as described in Legler et al., supra). The DNA coding for the different receptors was introduced in the pSG5 expression plasmid (Green et al., Nucleic Acid Res. 16: 369-369, 1988).

In order to determine the responsiveness of the U2-OS based steroid reporter cell lines, cells were plated in 96-wells plates and treated for 24 hours with hormone in the culture medium containing 5% charcoal-stripped serum. The results are shown in FIG. 2 (each point represents the mean of three independent experiments±SEM.

AR Cell Line

An AR cell line was generated by stably transfecting the human U2-OS cell line with pSG5-hAR and the 3×HRE-TATA-Luc reporter constructs. This stable cell line was characterized by its response to different steroids, as well as its non-responsiveness to other nuclear hormone receptor ligands. In contrast to other existing androgen receptor gene assays (Blankvoort et al., Ann. Biochem. 298: 93-102, 2001; Vinggaard et al., Toxicol. Appl. Pharmacol. 155: 150-160, 1999; Wilson et al., Toxicol. Sci. 66: 69-81, 2002; Terouanne et al., Mol. Cell. Endocrinol. 160: 39-49, 2000) the AR cell line according to the invention proved to excel in terms of cell line maintenance, inducibility of luciferase activity, minimal detection limit and androgen specificity. FIG. 2B shows the responsiveness of this cell line. The sequence of the androgen receptor used has been described by Trapman et al., BBRC 153: 241-248, (1988).

GR Cell Line

The GR cell line was generated by stably transfecting the human U2-OS cell line with pSG5-hGR and the 3×HRE-TATA-Luc reporter constructs. This stable cell line was characterized by its response to different steroids, as well as its non-responsiveness to other nuclear hormone receptor ligands. Compared to a 293-based cell line this cell line had a 10-fold lower EC50 value. This cell line was used to measure endogeneous corticosteroid activity levels in human blood samples. It was shown that this cell line is very suitable for measuring corticosteroid and synthetic glucocorticoid activity in serum. This assay can be carried out by adding serum directly to the cells. This type of application opens many possibilities and may also be applied to the other cell lines to measure hormonal activities of interest, for example total levels of estrogenic activity, which may be linked to the growth of children, or may be indicative for certain hormone related diseases. FIG. 2D shows the responsiveness of this cell line. The sequence of the glucocorticoid receptor used has been described by Hollenberg et al., Nature 318: 635-641, (1985).

FIG. 4 shows the results of the U2-OS cell line comprising glucocorticoid receptor in measuring diurnal endogenous cortisol levels in human serum, compared to measurements by cortisol specific RIA: A; GR reporter cells were plated in 96 well plates and treated for 24 hours with 5% (v/v) of human serum (collected from a healthy male volunteer at various daily time points) in culture medium containing 5% charcoal-stripped serum. Each point represents the mean of three independent experiments±SEM. B; Total cortisol levels as determined by radioimmunoassay in the same samples showing a similar diurnal pattern.

PR Cell Line

The PR cell line was generated by stably transfecting the human U2-OS cell line with pSG5-hPR and the 3×HRE-TATA-Luc reporter constructs. This stable cell line was characterized by its response to different steroids, as well as its non-responsiveness to other nuclear hormone receptor ligands. Compared to another existing progesterone receptor gene assay (Schoonen et al., J. Steroid Biochem. Mol. Biol. 64: 157-170, 1998) the PR cell line according to the invention proved to excel in terms of inducibility of luciferase activity. Moreover, this is the first human assay system. FIG. 2C shows the responsiveness of this cell line. The sequence of the progesterone receptor used has been described by Kastner et al., EMBO J. 9: 1603-1614, (1990).

ER-Alpha Cell Line

Two types of double transfectants were previously generated: 1. human ER-beta with the 3×HRE-TATA-Luc reporter construct; 2. Human ER-alpha with the 3×HRE-TATA-Luc reporter construct (Queadacker et al., Endocrinol. 142: 1156-1166, 2001). These cell lines are as responsive as the highly responsive 293-based cell lines (Lemmen et al., 2002) and have the additional advantage of a much better attachment to the cell culture plates making them much more easy to handle and suitable for high throughput screening. These cells are essential for the method according to the invention, i.e. for effect profiling, having the same cellular background as the above mentioned cell lines. FIG. 2A shows the responsiveness of the cell line comprising ER-alpha receptor. The sequence of the estrogen receptors used have been described by Green et al., Nature 320: 134-139, 1986 (ER-alpha) and Mosselman et al., FEBS Lett. 392: 49-53, 1996 (ER-beta).

### Example 2

Effect Profiling

The U2-OS cell line appears to be a very suitable cell line to be used in the method of the present invention, which may also be referred to as “effect profiling”. In the method, an array of cell lines is provided for a broad set of biological endpoints, for example all steroid receptor signalling pathways. The profile of activities of a sample measured in the different cell lines gives more relevant information on the presence of specific ligands in said sample, and in particular on the biological risks or benefits associated with said sample, the specificity of the response, and the nature of the biological active ligands in the sample, as compared to measurement of a single biological endpoint.

For example, metabolism of steroids can substantially alter their biological properties, and enzymatic conversions can lead to steroids with a totally different activity profile (FIG. 3). High concentrations of non-prohibited precursor steroids or precursor steroids that are difficult to trace are used in sport doping rather than the active hormone itself. When the precursor is converted into the active hormone at the level of the target cell, as often is the case, a mechanism of detection (either chemical of biological) directed towards the active circulating hormone will be ineffective. For instance, an androgen assay that does not detect these precursors may give a high level of false-negatives. With the method of the present invention precursor steroids can be detected by the so-called effect profiling. By providing an array of cell lines, for example comprising of a androgen-responsive cell line measuring the weakly androgenic androgen precursor androstenedione, but not DHEA, and an estrogen-responsive cell line measuring DHEA, but not androstenedione, the number of potential ligands that are measured by the method is increased. DHEA will therefore be measured with the method according to the invention.

An additional advantage of the method of the present invention is that if a ligand gives a generalized toxic response this will show up as a repression of all reporter gene assays, irrespective of the endpoint used. More specific toxic pathways can be identified by examining patterns of responses towards various reporter gene constructs. In the example above relating to the androgen precursors being active in one, but not in the other cell line it will be clear that this might be caused by specific conversions of the precursors. When the data from the array of cell lines are compared, specific biosynthetic routes can be deduced.

In addition, cellular activation signals leading to a variety of different responses, such as proliferation, apoptosis, DNA repair and differentiation, are activated through cellular pathways which may overlap (such as p53 activation, AP1 activation etc.) By analysing various signalling pathways at the same time in the array of cell lines, patterns will be revealed that will give information on the cellular fate and the relevance to other cellular systems.

Yet another example of the use of the method according to the invention is the detection of the synthetic progestin medroxyprogesterone actetate (MPA) which is illegal in the European Union as a growth promoter in animals, and is not allowed to be present in animal feed or their products. Contaminations of animal feed with this product has been found using expensive instrumental methods (Gass Chromatography/Mass Spectrometry; Van Leengoed et al., Tijdschrift Diergeneesk. 127: 516-519, 2002). Endogenous progestins such as progesterone are normally present in animal products such as meat, hampering detection with a simple single reporter gene assay responding on the net effect of all progestins present.

According to the present invention, however, it was found that MPA, in contrast to progesterone, also gives a considerable response in the U2-OS based AR and ERalpha cell lines, thereby facilitating its detection and identification when using all three cell lines. Other synthetic progestins such as Norethynodrel and Levonorgestrel also display different activity profiles compared to the endogenous ligand, progesterone (Table 2).

The data in Table 2 demonstrate that even with a limited set of cell lines the discrimination between different compounds can be improved. It will be clear that the discriminative power between compounds in an ‘effect profiling’ system will greatly improve by expanding the number of cell lines used. Automatisation of the handling is therefore an essential step in an efficient ‘effect profiling’ system. Obviously, the use of a single robust parent cell line, such as the U2-OS cells, with identical culture and handling conditions greatly facilitates the possibilities for automatisation.

TABLE 2
Data on potency (logEC50) of ligands. ND: not determined.

| | | | |
|---|---|---|---|
| Steroid | ER-alpha | AR | PR |

progestins

| | | | |
|---|---|---|---|
| progesterone | >−5.0 | −6.7 | −9.0 |
| MPA (synthetic) | −5.5 | −8.2 | −10.0 |
| Norethynodrel (synthetic) | −9.7 | −8.2 | −9.3 |
| Levenorgestrel (synthetic) | ND | −8.6 | −10.1 |

adrenal androgens

| | | | |
|---|---|---|---|
| DHEA | −7.6 | >−5.0 | >−5.0 |
| androstenedione | >−5.0 | −8.5 | ND |

androgens

| | | | |
|---|---|---|---|
| testosterone | −5.7 | −9.2 | −5.1 |
| dihydro-testosterone | −6.8 | −9.8 | −5.4 |
| Nandrolone (synthetic) | −7.4 | −9.5 | −8.3 |
| R1881 (synthetic) | >−5.0 | −9.8 | −7.6 |
| Cyproterone acetate (anti-anrogen | ND | <−5.0 | −9.1 |

estrogens

| | | | |
|---|---|---|---|
| 17β-estradiol | −11.6 | −5.5 | >−5.0 |
| 17α-estradiol | −9.3 | >−5.0 | ND |
| ethynyl-estradiol (synthetic) | −11.7 | >−5.0 | ND |

corticosteroids

| | | | |
|---|---|---|---|
| Dexamethasone | >−5.0 | >−5.0 | >−5.0 |

1. Method for determining the presence of one or more specific ligands in a sample, said method comprising;
a) contacting the sample with an array of cell lines, each cell line comprising a reporter gene construct responding to a cellular pathway which is induced by a different specific ligand;
b) measuring the activity of the reporter gene in the individual cell lines;
c) comparing the measured activity in the individual cell lines; and
d) determining the presence of the ligands in the sample based on said comparison.

2. The method as claimed in claim 1, wherein the cell lines originate from one parent cell line.

3. The method as claimed in claim 2, wherein the cell lines originate from the human osteoblastic cell line U2-OS.

4. The method as claimed in claim 1, wherein the array comprises at least two cell lines, preferably at least three cell lines.

5. The method as claimed in claim 1, wherein one or more of the cell lines comprise one or more expression plasmids each coding for a specific component of the cellular pathway.

6. The method as claimed in claim 5, wherein the specific component is a hormone receptor.

7. The method as claimed in claim 6, wherein the hormone receptor is a steroid hormone receptor or thyroid hormone receptor.

8. The method as claimed in claim 6, wherein the reporter gene construct comprises DNA coding for an operative hormone responsive element linked to a promoter and a reporter gene.

9. The method as claimed in claim 8, wherein the reporter gene construct comprises 3 tandem repeats of the hormone responsive element (HRE) oligonucleotide: AAGCTTAGAACAGTTTGTAACGAGCTCGTTACAAACTGTTCTAGCTCGTTACAAACTGTTCTAAGCTCAAGCTT (SEQ ID NO. 1) upstream of the minimal adenovirus E1B TATA promoter sequence (GGGTATATAAT) (SEQ ID NO. 2) inserted in the multiple cloning site of the luciferase reporter construct pGL3.

10. The method as claimed in claim 8, wherein the DNA coding for the different hormone receptors is introduced in the pSG5 expression plasmid.

11. The method as claimed in claim 5, wherein the specific component is a ligand modifying factor.

12. The method as claimed in claim 11, wherein the ligand modifying factor is an enzyme.

13. Human osteoblastic cell line U2-OS, comprising a reporter gene construct comprising DNA coding for an operative hormone responsive element linked to a promoter and a reporter gene, and one or more expression plasmids comprising DNA coding for a hormone receptor, wherein the hormone receptor is selected from the group consisting of androgen receptor, progesterone receptor, glucocorticoid receptor, mineralocorticoid receptor, and thyroid receptor.

14. Human osteoblastic cell line as claimed in claim 13, wherein the reporter gene construct comprises 3 tandem repeats of the hormone responsive element (HRE) oligonucleotide: AAGCTTAGAACAGTTTGTAACGAGCTCGTTACAAACTGTTCTAGCTCGTTACAAACTGTTCTAAGCTCAAGCTT (SEQ ID NO. 1) upstream of the minimal adenovirus E1B TATA promoter sequence (GGGTATATAAT) (SEQ ID NO. 2) inserted in the multiple cloning site of the luciferase reporter construct pGL3.

15. Human osteoblastic cell line as claimed in claim 13, wherein the DNA coding for the different hormone receptors is introduced in the pSG5 expression plasmid.

16. Use of human osteoblastic cell lines in an assay for determining the presence of one or more ligands in a sample.

17. Use as claimed in claim 16, wherein the cell line is the U2-OS cell line.

18. The method as claimed in claim 2 wherein the array comprises at least two cell lines, preferably at least three cell lines.

19. The method as claimed in claim 2, wherein one or more of the cell lines comprise one or more expression plasmids each coding for a specific component of the cellular pathway.

20. The method as claimed in claim 3, wherein the array comprises at least two cell lines, preferably at least three cell lines.

21. The method of claim 3, wherein one or more of the cell lines comprise one or more expression plasmids each coding for a specific component of the cellular pathway.

22. The method of claim 4 wherein one or more of the cell lines comprise one or more expression plasmids each coding for a specific component of the cellular pathway.

23. The method of claim 7, wherein the reporter gene construct comprises DNA coding for an operative hormone responsive element linked to a promoter and a reporter gene.

24. The method of claim 9, wherein the DNA coding for the different hormone receptors is introduced in the pSG5 expression plasmid.

25. Human osteoblastic cell line as claimed in claim 14 wherein the DNA coding for the different hormone receptors is introduced in the pSG5 expression plasmid.


SEQUENCE LISTING (2 sequences)

SEQ ID NO: 1 - DNA, 74 bp, Artificial Sequence
  Synthetic hormone responsive element.
aagcttagaacagtttgtaacgagctcgttacaaactgttctagctcgttacaaactgttctaagctcaagctt

SEQ ID NO: 2 - DNA, 11 bp, Artificial Sequence
  Synthetic adenovirus E1B TATA promoter sequence.
gggtatataat